SEQUENCE LISTING (8 sequences)

SEQ ID NO: 1 - DNA, 20 bp, Artificial
  Primer
ttcgccgaccgttgactatt

SEQ ID NO: 2 - DNA, 23 bp, Artificial
  Primer
aagattattacaaatgcatgggc

SEQ ID NO: 3 - DNA, 22 bp, Artificial
  Primer
gccagccaggcaaccttctagg

SEQ ID NO: 4 - DNA, 29 bp, Artificial
  Primer
aacgaccacatctacaacgttatcgtcac

SEQ ID NO: 5 - DNA, 20 bp, Artificial
  Primer
gagctgttgacggaaaggag

SEQ ID NO: 6 - DNA, 21 bp, Artificial
  Primer
cagaagagaatcccggctaag

SEQ ID NO: 7 - DNA, 27 bp, Artificial
  Primer
gaggcgctgttagagatctgtcagaga

SEQ ID NO: 8 - DNA, 25 bp, Artificial
  Primer
ggcatttcctaagtggaagcaagca

(19) United States
(12) Patent Application Publication
(10) Pub. No.: US 2009/0181367 A1
(43) Pub. Date: Jul. 16, 2009

(54) Diagnosis of sepsis using mitochondrial nucleic acid assays

(76) Inventors: Helene Cote, Vancouver (CA); Julio Montaner, Vancouver (CA); Michael V. O'Shaughnessy, Maple Ridge (CA)

Correspondence Address: Jeffrey J. King, Esq.;BLACK LOWE & GRAHAM PLLC, 701 Fifth Avenue - Suite 4800, Seattle, WA, 98104, US

(21) Appl. No.: 11/900,918

(22) Filed: Sep. 12, 2007

Related U.S. Application Data
(63) Continuation of application No. 11/030,602, filed on Jan. 5, 2005, now abandoned.
Application No. 11/030,602 is a continuation of application No. PCT/CA03/01006, filed on Jul. 4, 2003.
(60) Provisional application No. 60/393,386, filed on Jul. 5, 2002.

(51) Int. Cl. C12Q 1/68

(52) U.S. Cl. 435/6

(57) ABSTRACT

The invention provides assays to detect sepsis disease states in a subject by determining the relative amount of mitochondrial nucleic acid in the subject. The assays of the invention may include PCR assays, such semi-quantitative or quantitative PCR involving the co-amplification of a mitochondrial sequence and a reference sequence, such as a genomic sequence. Information from such assays may be evaluated to provide a ratio of mitochondrial nucleic acid to nuclear nucleic add in the cells of the subject.

### FIELD OF THE INVENTION

The invention is in the field of diagnostic or prognostic assays for sepsis.

### BACKGROUND OF THE INVENTION

Sepsis is a potentially life-threatening, systemic clinical condition that can develop after infection or traumatic injury (Mesters, R. M. et al. (1996) Thromb Haemost. 75:902-907; Wheeler A. P. and Bernard G. R. (1999) N Engl J Med 340:207-214). Generally, sepsis is thought to be caused by the release of microorganism toxins during severe infection, although a septic response can also result from other conditions including surgery, physical trauma, burn injuries, organ transplantation, or pancreatitis, in the absence of any indication of a concomitant microbial infection (Balk R. A. and Bone R. C. (1989) Crit Care Clin 5:1-8; Ayres S. M. (1985) Crit Care Med 13:864-66). In humans, release of endotoxins derived from the lipopolysaccharide outer membrane of virtually all gram-negative bacteria is thought to be a common cause of sepsis.

The sequelae of sepsis may be characterized by severe hypotension, sequential multiple organ failure or dysfunction, and necrotic cell death, and can be the most frequent cause of mortality in intensive care units, and may result in severe sepsis, sepsis-induced hypotension, or septic shock (Parrillo, J. E. et al. (1990) Ann Intern Med 113:22742; Manship, L. et al. (1984) Am Surg 50:94-101; Niederman M. S. and Fein A. M. (1990) Clin Chest Med 11:663-65). Despite advances in medicine and critical care management protocols, patients who go into septic shock have an estimated mortality ranging from 30% to 50%, depending on the presence of other medical complications (Natanson, C. et al. (1998) Crit Care Med. 26:1927-1931; Zeni, F. et al. (1997) Crit Care Med. 25:1095-1100).

The timing of treatment protocols for sepsis may be critical to successful outcomes. Delay in initiation of treatment can have severe consequences for the patient. Additionally, the optimal window of administration for a therapeutic agent may depend on the stage to which the sepsis has progressed. Therefore, rapid and reliable diagnosis of sepsis is key to effective intervention.

Present diagnostic techniques for sepsis include positive blood tests for microorganisms or acidosis; tests for alterations in white blood cell or platelet count; or identification of physical symptoms such as fever, chills, shaking, hyperventilation, increased heart beat, confusion or delirium (von Landenberg, P and Shoenfeld, Y (2001) IMAJ 3: 439-442; Marshall, J. C et al. (1995) Crit Care Med 23 (10):1638-1652; Vincen, J. L et al. (1996) Intensive Care Med 22:707-710; Le Gall, J. R. et al. (1996) JAMA 276:802-810; Knaus, W. A. et al. (1985) Crit Care Med 13:818-829). These diagnostic techniques however may be of limited value since the results of blood cultures may arrive too late for successful intervention, or may not be sufficiently sensitive, and the physical symptoms of sepsis may be attributable to other conditions, leading to inappropriate treatment.

It would be useful to develop rapid and reliable diagnostic tests capable of identifying patients early in the progression of sepsis, generally prior to the development of septic shock, to monitor the condition of a patient undergoing treatment for sepsis, or to identify patients who are at risk for developing sepsis. It would also be useful to develop diagnostic tests to facilitate the discovery and development of new therapeutics for sepsis.

### SUMMARY OF THE INVENTION

In various alternative aspects, the invention provides methods for the detection of symptoms in sepsis patients based, in general, on the discovery that mitochondrial nucleic acids (for example, mitochondrial DNA or RNA, such as mitochondrial mRNA ) may be depleted in subjects having sepsis. In some patients, the methods may be useful for detecting sepsis generally, regardless of the underlying cause of sepsis.

In one aspect, the invention provides a method of diagnosis of a sepsis disease state in a subject in need of such diagnosis. The method includes determining the relative amount of mitochondrial nucleic acid in a sample from the subject, where the determined relative amount of mitochondrial nucleic acid may be indicative of the presence of the sepsis disease state in the subject. In another aspect, the invention provides a method of predicting the risk for a sepsis disease state in a subject in need of such prediction. The method includes determining the relative amount of mitochondrial nucleic acid in a sample from the subject, where the determined relative amount of mitochondrial nucleic acid may be indicative of the risk for the sepsis disease state in the subject. In either of these aspects, the subject may be suffering from sepsis.

In another aspect, the invention provides a method of monitoring the progression of a sepsis disease state in a subject having sepsis. The method includes determining the relative amount of mitochondrial nucleic acid in samples from the subject at first and second time points, where the difference between the determined relative amount of mitochondrial nucleic acid between the first time point and the second time point may be indicative of the progression of the sepsis disease state in the subject. The method may also include determining the relative amount of mitochondrial nucleic acid in a sample from the subject at subsequent time points, or include prognosing or predicting the likely clinical course of sepsis in the subject.

In another aspect, the invention provides a method of determining the efficacy of a therapy for sepsis. The method includes determining the relative amount of mitochondrial nucleic acid in a sample from a control subject and from a test subject, where the test subject is administered the therapy, and where the difference between the determined relative amount of mitochondrial nucleic acid in the sample from the test subject and the control subject may be indicative of the efficacy of the therapy.

In an alternative aspect, the invention provides a diagnostic kit for use in determining the extent of a sepsis disease state in a subject by determining the relative amount of mitochondrial nucleic acid in a sample from the subject. The kit may include a mitochondrial nucleic acid primer and a nuclear nucleic acid primer.

In alternative embodiments, the subject may be a human (e.g., a neonate, an elderly individual, or an immunocompromised individual) or a non-human animal (e.g., an animal model of sepsis, such as LPS-induced sepsis). In some embodiments, the subject may be diagnosed with sepsis or determined to be at risk for sepsis, or a treatment for sepsis may be initiated in the subject, if the relative amount of mitochondrial nucleic acid falls below a predetermined level. The predetermined level may be, optionally, expressed as a ratio of, for example, mitochondrial DNA (mtDNA) to nuclear DNA (nDNA) with reference to a standard mtDNA/nDNA ratio set at 1, where the predetermined level may be a ratio of 0.45 or less.

In other alternative embodiments, the sepsis disease state may be a sepsis symptom resulting from a gram negative bacterial infection, gram positive bacterial infection, fungal infection, viral infection, physical trauma, pancreatitis, organ transplantation, hemorrhage, adult respiratory distress syndrome, burn injury, surgery, chemotherapy, or exposure to ionizing radiation. The sample may, for example, be a peripheral blood sample.

In alternative embodiments, the relative amount of mitochondrial nucleic acid may indicate the severity of sepsis or the success of a therapeutic treatment for sepsis. The mitochondrial nucleic acid may be determined relative to the amount of nuclear nucleic acid in the cells of the subject, for example, by a polymerase chain reaction, such as a quantitative polymerase chain reaction, where amplification of the mitochondrial nucleic acid may be compared to amplification of a reference nucleic acid. The polymerase chain reaction may be a real-time polymerase chain reaction where an amplification product may be detected with a hybridization probe.

### DETAILED DESCRIPTION OF THE INVENTION

“Sepsis,” as used in the context of the present invention, includes the terms “sepsis,” “bacteremia,” “septicemia,” “septic syndrome,” “septic shock,” “severe sepsis,” and “systemic inflammatory response syndrome” or “SIRS,” and refers, in general, to an acute systemic inflammatory reaction, associated with the release of endogenous mediators of inflammation into the bloodstream, such as proinflammatory cytokines, adhesion molecules, vasoactive mediators, and reactive oxygen species, and accompanied by altered white blood cell count, body temperature, heartbeat, and respiration (Bone, R. C. et al. (1992) Chest 101:1644-55; Paterson, R. L., and N. R. Webster (2000) J. R. Coll. Surg. Edinb., 45: 178-182). Sepsis is generally thought to be triggered by infection or injury, and is a non-specific host response to infectious microorganisms, including gram-negative and gram-positive bacteria, fungi, protozoa, and viruses; or to inflammation mediators resulting from infection or injury (Bone, R. C. et al. (1992) Chest 101:1644-55; Paterson, R. L., and N. R. Webster (2000) J. R. Coll. Surg. Edinb., 45: 178-182).

In some embodiments, SIRS may be diagnosed in patients having two or more of the following indications: body temperature greater than 38° C. or less than 36° C.; tachycardia greater than 90 beats/minute; respiratory rate greater than 20 breaths/minute or PaCO2 less than 4.3 kPa; and white blood count greater than 12×10⁹/l or less than 4×10⁹/l or greater than 10% immature (band) forms. In alternative embodiments, sepsis may be defined as SIRS due to infection, and severe sepsis may be defined as sepsis with evidence of organ hypoperfusion. In alternative embodiments, septic shock may be defined as severe sepsis with hypotension (systolic BP less than 90 mmHG) despite adequate resuscitation or the requirement for vasopressors/inotropes to maintain blood pressure (Paterson, R. L., and N. R. Webster (2000) J. R. Coll. Surg. Edinb., 45: 178-182). Sepsis, if not diagnosed and treated in time, can develop into septic shock, which can result in a life-threatening drop in blood pressure, and the inflammation-related effects of sepsis may lead to tissue injury and to progressive organ dysfunction and organ failure.

Sepsis can result from a local infection in organs such as the kidneys (e.g., due to an upper urinary tract infection); liver; gall bladder; bowel (e.g., peritonitis); skin (e.g., cellulitis); lungs (e.g., bacterial pneumonia); the genitourinary tract (e.g., urosepsis); or brain and spinal cord (e.g., bacterial meningitis), or can result from a systemic condition such as toxic shock syndrome. Sepsis can also occur as a result of non-infectious insults such as chemotherapy, acute pancreatitis, surgery, physical trauma, burn injuries, organ transplantation, multiple organ dysfunction syndrome (MODS), acute respiratory distress syndrome (ARDS), acute lung injury (ALI), disseminated intravascular coagulation (DIC), hemorrhage, or exposure to ionizing radiation (Bone, R. C. et al. (1992) Chest 101:1644-55; Paterson, R. L., and N. R. Webster (2000) J. R. Coll. Surg. Edinb., 45: 178-182).

The physiological symptoms of sepsis occur on a continuum that ranges from shaking, chills, fever, weakness, nausea, vomiting, and diarrhoea to the severe hypotension, reduced mental alertness and confusion, sequential multiple organ failure or dysfunction (for example, of the kidneys, causing low urine output; the lungs, causing breathing difficulties and low levels of oxygen in the blood; the heart, causing fluid retention and swelling), and necrotic and apoptotic cell death that is characteristic of septic shock.

In alternative embodiments, the methods of the invention include the quantification of mitochondrial nucleic acid, such as mitochondrial DNA (mtDNA) or mitochondrial RNA, e.g., mitochondrial mRNA (mt mRNA) in a sample, such as a peripheral blood sample or a cellular fraction thereof, from a subject, to determine whether the mitochondrial nucleic acid levels are at levels indicative of sepsis. A “sample” can include any biological fluid, cell, or tissue, including without limitation, peripheral blood, lymphocytes (e.g., B cells, CD4 T cells, CD8 T cells), sputum, urine, wounds, entrance sites for catheters from a subject, or cell lines derived thereof. In alternative aspects of the invention, samples for use in the assays of the invention may be obtained, for example by autopsy or biopsy, from a variety of tissues, such as from heart, brain, lung, kidney, fat, spleen, or liver, or cells derived therefrom.

In alternative embodiments, the methods of the invention also include assays to determine the relative amount of mitochondrial nucleic acid in a subject, such as a subject suspected of having sepsis or at risk for sepsis. The subject may for example be a human patient undergoing treatment for an acute infection, or may be a member of a group vulnerable to sepsis. In some embodiments, the subject may be a non-human animal, for example, a domestic or farm animal, such as a dog, pig, sheep, cow, chicken, or turkey. In some embodiments, the subject may be an animal model of sepsis, as known to those of skill in the art or as described herein, and may be a rat, mouse, sheep, pig, baboon, rhesus monkey, or dog.

The assays of the invention can include PCR assays, such semi-quantitative or quantitative PCR or RT PCR involving the co-amplification of a mitochondrial sequence and a reference sequence, such as a genomic sequence. The assays of the invention can also include hybridization assays, for example, RNA or DNA hybridization assays, using mitochondrial and nuclear DNA or RNA samples and mitochondrial and reference (e.g. genomic or cDNA) sequences as probes. Information from such assays can be evaluated to provide a ratio of mitochondrial nucleic acid to nuclear nucleic acid (e.g, mt DNA to n DNA or mt mRNA to nuclear RNA (nRNA)) in the cells or tissues of the subject.

In various embodiments of the invention, the assays could therefore provide clinical information before sepsis develops or becomes severe enough to approach septic shock. The depletion in mitochondrial nucleic acid (e.g., mtDNA or mt RNA) may be reversed upon administration of a suitable therapy, for example, a suitable broad spectrum antibiotic. Severe symptoms of sepsis, including septic shock, may occur when the mitochondrial nucleic acid (e.g., mtDNA or mt RNA) levels fall below approximately any value from 50%, 40%, 30%, 20%, or 10% of normal, as measured by reference to a control sample or to a known standard. Clinical intervention for sepsis may also be indicated when mitochondrial nucleic acid to nuclear nucleic acid ratios (for example, mtDNA to nDNA ratios or mt mRNA to nRNA ratios) fall below a threshold value such as 0.5, 0.45, 0.4, 0.35 or 0.3., as measured with respect to a control sample or to a known standard.

In alternative embodiments, the rate of change of relative mitochondrial nucleic acid (e.g., mtDNA or mt RNA) concentration over a time period may also be determined to provide diagnostic information. For some subjects, a relatively rapid decrease in the relative amount of mitochondrial nucleic acid (e.g., mtDNA or mt RNA) may be indicative of sepsis. A relatively rapid decrease of on the order of 50% or more (or more than 40% in some cases) in the relative amount of mitochondrial nucleic acid compared to nuclear nucleic acid (for example, mtDNA compared to nDNA or mt mRNA compared to nDNA), over a period of less than eight to ten days may indicate that a subject is developing sepsis, and may therefore need to be monitored more closely, and/or may need to be administered antibiotics or other anti-sepsis therapeutics.

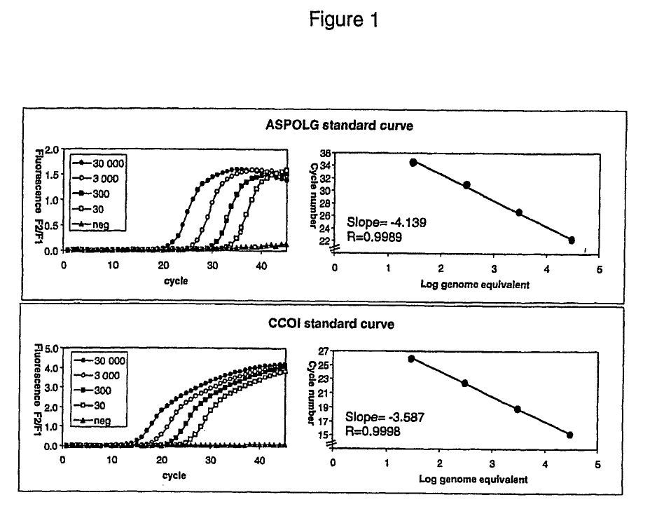

In alternative embodiments, the invention also provides protocols that, for example, avoid the necessity to determine mtDNA copy number per se, facilitating instead a determination of the relative amount of mitochondrial nucleic acid (for example, mtDNA or mt mRNA), for example, the amount relative to nuclear nucleic acid (for example, nDNA or nRNA) sequence. In some aspects, this approach may simplify the diagnostic assays of the invention. For example, as shown in FIG. 1, numbers (30 to 30,000) representing nuclear-genome-equivalents are assigned to nDNA amplification standards, as determined by calibration with a control human DNA of known nuclear-genome-equivalent concentration. The same numbers are arbitrarily assigned to the corresponding standard curves for the mitochondrial gene (although they do not represent a calculated copy number of the mitochondrial gene). In an alternative approach, the numbers representing nuclear-genome-equivalents may be arbitrarily assigned to, for example, the nDNA amplification standards, based only on the degree of sample dilution (so that the number reflect the relative copy number of nuclear-genome-equivalents, but not the absolute value of such equivalents), and these arbitrary numbers may similarly be assigned to the mtDNA amplification standards. The results of the assays of the invention may then be expressed by the ratio of, for example, mtDNA to nDNA, without the need to determine absolute mtDNA copy numbers. In such embodiments, it may be preferable to utilize an initial concentration of sample DNA or RNA that provides sufficient PCR template so that the number of amplification cycles is within the range which provides the most reliable results, such as from a minimum of any integer from 5 to 15 up to a maximum of any integer from 15 to 40.

The invention therefore provides a process for comparing the relative abundance of nucleic acid sequences, including:

a) measuring the amplification kinetics of a nuclear DNA or RNA sequence under a nuclear amplification reaction condition in a first nuclear control sample and in a second nuclear control sample, to obtain control nuclear amplification measurements, wherein the first and the second nuclear control samples have different concentrations of the nuclear DNA or RNA sequence;

b) constructing a control nuclear DNA or RNA sequence dataset from the control nuclear amplification measurements, to obtain a model standard relationship between amplification kinetics and concentration for the nuclear DNA or RNA sequence;

c) measuring the amplification kinetics of a mitochondrial DNA or RNA sequence under a mitochondrial amplification reaction condition in a first mitochondrial control sample and in a second mitochondrial control sample, to obtain control mitochondrial amplification measurements, wherein the first and the second mitochondrial control samples have different concentrations of the mitochondrial DNA or RNA sequence;

d) constructing a control mitochondrial DNA or RNA sequence dataset from the control mitochondrial amplification measurements, to obtain a model standard relationship between amplification kinetics and concentration for the mitochondrial DNA or RNA sequence;

e) measuring the amplification kinetics of the nuclear DNA or RNA sequence under the nuclear amplification reaction conditions in a test sample, to obtain a test sample nuclear amplification measurement;

f) applying the model standard relationship between amplification kinetics and concentration for the nuclear DNA or RNA sequence to the test sample nuclear amplification measurement, to obtain a test sample nuclear DNA or RNA sequence concentration measurement;

g) measure the amplification kinetics of the mitochondrial DNA or RNA sequence under the mitochondrial amplification reaction conditions in the test sample, to obtain a test sample mitochondrial amplification measurement;

h) applying the model standard relationship between amplification kinetics and concentration for the mitochondrial DNA or RNA sequence to the test sample mitochondrial amplification measurement, to obtain a test sample mitochondrial DNA or RNA sequence concentration measurement;

i) comparing the test sample nuclear DNA or RNA sequence concentration measurement to the test sample mitochondrial DNA or RNA sequence concentration measurement, to determine the relative concentration of the mitochondrial DNA or RNA sequence compared to the nuclear DNA or RNA sequence in the test sample.

In alternative embodiments, the methods and kits of the invention can be used to identify those individuals among the vulnerable groups who are at a greater risk of acute infection, and as a result, sepsis. For example, neonates i.e., newborn infants or the fetus, and very young children (under the age of two years) are particularly susceptible to infections leading to sepsis, as are the elderly and people who are immunocompromised (including people subjected to severe physical trauma, such as burn patients). In some embodiments of the invention, individuals diagnosed with, suspected of having, or at risk for HIV infection or cancer are excluded from the methods of the invention, so that the invention includes methods of determining the relative amount of mitochondrial nucleic acid in a sample from a patient that is not an individual diagnosed with, suspected of having, or at risk for HIV infection or cancer. In some embodiments, the methods and kits of the invention may be used to identify or monitor a sepsis disease state in a non-human animal, for example, a domestic, farm, or experimental animal.

Present interventions include subjecting a vulnerable individual with a high temperature to treatment with broad spectrum antibiotics or to lumbar puncture, to rule out meningitis. Thus, under the present sepsis management schemes, large numbers of patients who do not have sepsis are unnecessarily treated against sepsis, which is undesirable for many reasons. For example, administration of broad spectrum antibiotics is undesirable due to the risks associated with antibiotic therapy, for example, drug allergy, hearing loss, or damage to internal organs due to poor clearance of the drug, and to the development of antibiotic resistance strains of bacteria, while procedures like lumbar puncture are relatively invasive and cause more trauma to the patient.

Using the rapid, and relatively noninvasive methods of the invention, interventions could be restricted to those patients who are identified as having sepsis. Such patients may be treated with infection-specific therapeutics, if available; may be treated with broad spectrum antibiotics earlier or more aggressively; or may be subjected to procedures like lumbar puncture. Unlike sepsis detection methods that rely on the presence of microorganisms in the bloodstream, the methods of the invention may be used to detect sepsis may for example be undertaken when the subjects are treated with an antisepsis drug, such as a new antibiotic.

In alternative embodiments, the methods of the invention may also be used to identify vulnerable individuals and individuals with sepsis who would benefit from early intervention. This identification can assist a health care practitioner to undertake early or perhaps more aggressive therapies. Thus, a patient showing severely depleted relative mitochondrial nucleic acid levels, for example, mtDNA or mt RNA levels, may merit aggressive, continuous and/or multiple antibiotic treatment. Similarly, it may be advisable to postpone surgery in a patient with depleted relative mitochondrial nucleic acid levels, for example, mtDNA or mt RNA levels.

In alternative aspects, the methods of the invention may be used, for example, in experimental models of sepsis, to test the efficacy of new therapies for the treatment of sepsis. Animal models of sepsis include, without limitation: administration of bacterial endotoxin (lipopolysaccharide, LPS) to simulate sepsis caused by gram negative bacteria; chemotherapy-induced infection; cecal ligation and double puncture in rats, to model the acute respiratory distress syndrome; or any other experimental model that can simulate the symptoms of sepsis. Description of animal models of sepsis may be found in, without limitation, Bhatti A R and Micusan V V (1996) Microbios 86 (349):247-53; Redl H, et al. (1993) Immunobiology 187 (3-5):330-45; Fink M P and Heard S O (1990) J Surg Res. 49 (2):186-96; Dunn D L (1988) Transplantation 5 (2):424-9; Bohnen J M, et al. (1988) Can J Microbiol. 34 (3):323-6; Mela-Riker L, et al (1988) Circ Shock. 25 (4):231-44; Walker J F, et al. (1986) Am J Kidney Dis. 8 (2):88-97; Lopez-Garrido J, et al. (1987) Lab Invest. 56 (5):534-43; Quimby F and Nguyen H T (1985) Crit Rev Microbiol. 12 (1):144; Noel G J, et al. (1985) Pediatr Res. 19 (3):297-9; Moesgaard F, et al. (1983) Eur J Clin Microbiol. 2 (5):459-62; Hinshaw L B, et al. (1976) Surg Gynecol Obstet. 142 (6):893-900; Tieffenberg J., et al. (1978) Infect Immun. 19 (2):481-5; Wing D. A., et al. (1978) J Lab Clin Med. 92 (2):239-51, all of which are incorporated by reference herein.

In alternative aspects, the invention provides kits having components for use in methods of the invention. Such kits may comprise PCR components, as set out in detail below, including PCR primers specific for a mtDNA or mtRNA sequence and for a nDNA or nRNA sequence. Such kits may also include written instructions for carrying out the methods of the invention as described herein.

In alternative embodiments, a variety of techniques may be used to measure the relative amount of a mitochondrial DNA or RNA in cells. Methods of quantitative PCR are for example disclosed in the following documents, all of which are incorporated herein by reference: U.S. Pat. No. 6,180,349 issued to Ginzinger, et al. Jan. 30, 2001; U.S. Pat. No. 6,033,854 issued to Kurnit, et al. Mar. 7, 2000; and U.S. Pat. No. 5,972,602 issued to Hyland, et al. Oct. 26, 1999; Song, J. et al. (2001) Diabetes Care 24:865-869. A mitochondrial DNA or RNA sequence may be chosen from any mitochondrion-specific nucleotide sequence, including but not limited to ATP synthase 6, GenBank Accession No. AF368271; tRNA-Leu, GenBank Accession No. S49541; NADH dehydrogenase subunit 5 (MTND5), GenBank Accession No. AF339085; IDL, GenBank Accession No. AF079515; cytochrome b, GenBank Accession No. AF254896, CCOI, or any other suitable any mitochondrion-specific nucleotide sequence. A nuclear DNA or RNA sequence may be chosen from any sequence, including but not limited to a human 28S rRNA sequence, an ASPOL-gamma sequence, a beta-globin sequence, or any other suitable nuclear DNA or RNA sequence. Amplification probes may be designed according to methods known in the art and described, for example, in Sambrook, et al. (Molecular Cloning: A Laboratory Manual. 2^{nd}, ed., Cold Spring Harbor Laboratory, Cold Spring Harbor Laboratory Press, Cold Spring Harbor, N.Y., 1989) or Ausubel et al. (Current Protocols in Molecular Biology, John Wiley & Sons, 1994).

In alternative aspects, the methods of the invention may be used in conjunction with other therapeutic, preventative, or diagnostic methods for sepsis, including but not limited to those described in von Landenberg, P and Shoenfeld, Y (2001) IMAJ 3: 439-442; Marshall, J. C., Cook, D. J., Christou, N. V., Bernard, G. R., Sprung, C. L., Sibbald, W. J. (1995) Crit Care Med 23 (10):1638-1652; Vincent J. L., Moreno R., Takala J., Willats S., (1996) Intensive Care Med 22:707-710; Le Gall J. R., Klar J., Lemeshow S. (1996) JAMA 276:802-810; Knaus, W. A., Draper, E., Wagner, D., and Zimmerman, J. (1985) Crit Care Med 13:818-829; Knaus W. A., Wagner D. P., Draper E. A., Zimmerman J. E., et al. (1991) Chest 100: 1619-38; Bone, R. C., Balk, R. A., Cerra, F. B., Dellinger, R. P., Fein, A. M., Knaus, W. A., Schein, R. M. H., Sibbald, W. J. (1992) Chest 101:1644-55; U.S. Pat. No. 6,303,321, issued to Tracey, et al., Oct. 16, 2001; U.S. Pat. No. 5,993,811, issued to Becker, et al., Nov. 30, 1999; U.S. Pat. No. 5,830,679, issued to Bianchi, et al., Nov. 3, 1998; U.S. Pat. No.5,804,370, issued to Romaschin, et al., Sep. 8, 1998; U.S. Pat. No. 5,780,237, issued to Bursten, et al., Jul. 14, 1998; U.S. Pat. No. 5,639,617, issued to Bohuon, Jun. 17, 1997; U.S. Pat. No. 5,545,721, issued to Carroll, et al., Aug. 13, 1996; or U.S. Pat. No.5,998,482, issued to David, et al., Dec. 7, 1999, all of which are incorporated by reference herein. Alternatively or additionally, such patients may be treated with mitochondrial therapeutics, i.e. compositions of benefit to mitochondria, such as mitochondrial enzyme co-factors or precursors. In some embodiments, such mitochondrial therapeutics may for example be selected from the group consisting of riboflavin (vitamin B2), coenzyme Q10, vitamin B1 (thiamine), vitamin B12, vitamin K, 1-acetyl carnitine, N-acetyl cysteine and nicotinamide.

### EXAMPLE 1

Sepsis is associated with a significant decrease in blood cell mtDNA content. An assay is provided to monitor mitochondrial DNA levels, for example in subjects with sepsis. Methods of the invention may be adapted to assess the efficacy of anti-sepsis drugs and to diagnose sepsis in patients having sepsis or in individuals suspected to be at risk for sepsis.

Materials and Methods

Longitudinal blood samples can be collected from a subject. Total DNA can be extracted from blood cells and both a nuclear gene and a mitochondrial gene can be amplified and quantified by Real-Time PCR using hybridization probes. The mtDNA levels can be expressed as a ratio of the mitochondrial over nuclear DNA (mtDNA/nDNA).

Sample Collection and DNA Extraction

Buffycoats can be collected from blood samples and stored frozen at −70° C. until used. Total DNA can be extracted from 200 μL of buffycoat using the QIAamp DNA Blood Mini kit (QIAGEN, Missisauga, Ontario, Canada) according to the manufacturer's protocol, and resuspended in 200 μL of elution buffer. For the standard curves, similar samples can be collected from control volunteers and the DNA can be extracted and pooled. The nuclear genome equivalent (g.eq.) content of the DNA pool can be determined by calibration with control kit human DNA of known nuclear g.eq. concentration (Roche Molecular Biochemicals, Laval, Quebec, Canada).

Quantitative Real-Time PCR

For the mtDNA CCOI gene, the CCOI1F 5′-TTCGCCGACCGTTGACTATT-3′ (SEQ ID NO: 1) and CCOI2R 5′-AAGATTATTACAAATGCATGGGC-3′(SEQ ID NO: 2) primers can be used for the PCR amplification and the oligonucleotides 3′-Fluorescein-labeled CCOIPR1 5′-GCCAGCCAGGCAACCTTCTAGG-F-3′ (SEQ ID NO: 3) and 5′LC Red640-labeled CCOIPR2 5′-L-AACGACCACATCTACAACGTTATCGTCAC-P-3′(SEQ ID NO: 4), the 3′ end of the latter blocked with a phosphate molecule, can be used as hybridization probes.

For the nDNA ASPOLγ gene, the ASPG3F 5′-GAGCTGTTGACGGAAAGGAG-3′ (SEQ ID NO: 5) and ASPG4R 5′-CAGAAGAGAATCCCGGCTAAG-3′ (SEQ ID NO: 6) primers can be used for the PCR and the oligonucleotides 3′-Fluorescein-labeled ASPGPR1 5′-GAGGCGCTGTTAGAGATCTGTCAGAGA-F-3′ (SEQ ID NO: 7) and 5′LC Red640-labeled, 3′-Phosphate-blocked ASPGPR2 5′-L-GGCATTTCCTAAGTGGAAGCAAGCA-P-3′ (SEQ ID NO: 8) can be used as hybridization probes.

The real-time PCR reactions can be done separately and in duplicate for each gene, using the LightCycler FastStart DNA Master Hybridization Probes kit (Roche Molecular Biochemicals, Laval, Quebec, Canada). The PCR reactions can contain 5 mM MgCl₂, 0.5 μM of each primer, 0.1 μM 3′-Fluorescein probe, 0.2 μM 5′LC Red640 probe and 4 μL of a 1:10 dilution of the DNA extract in elution buffer. The PCR amplification can consist of a single denaturation/enzyme activation step of 10 min at 95° C. followed by 45 cycles of 0 s/95° C., 10 s/60° C., 5 s/72° C., with a 20° C./s temperature transition rate. The gain settings can be F1=1, F2=8 and a single fluorescence acquisition can be made at the end of each annealing step. An external standard curve of 30, 300, 3000, and 30000 nuclear g.eq. can be included in each LightCycler run, and the same nuclear g.eq values were used for both the nuclear (ASPOLγ) and the mitochondrial (CCOI) genes. The data can be analyzed using the second derivative maximum of each amplification reaction and relating it to its respective standard curve. Results from the quantitative PCR can be expressed as the relative ratio of the mean mtDNA g.eq. of duplicate measurements over the mean nDNA g.eq. of duplicate measurements for a given extract (mtDNA/uDNA), a ratio arbitrarily set around 1.0 by the fact that the same nuclear g.eq. values can be used to generate both standard curves.

In some embodiments, PCR methods of the invention may be real-time polymerase chain reactions wherein an amplification product is detected with a hybridization probe, such as described above using the LightCycler FastStart DNA Master Hybridization Probes kit (Roche Molecular Biochemicals, Laval, Quebec, Canada) or alternative commercially available techniques such as ABI Taqman® technology (using for example an ABI Prism 7700 instrument to detect accumulation of PCR products continuously during the PCR process, Applied Biosystems, Foster City, Calif., U.S.A.). Alternative PCR methods and variations on the forgoing methods may be adopted, as for example are disclosed in the following U.S. patents which are hereby incorporated by reference: U.S. Pat. No. 6,180,349 (Ginzinger et al. Jan. 30, 2001); U.S. Pat. No. 6,033,854 (Kuit et al. Mar. 7, 2000); U.S. Pat. No. 5,972,602 (Hyland; Oct. 26, 1999); U.S. Pat. Nos. 5,476,774 and 5,219,727 (Wang; Dec. 19, 1995 and Jun. 15, 1993); U.S. Pat. No. 6,174,670 (Wittwer et al. Jan. 16, 2001); U.S. Pat. No. 6,143,496 (Brown; Nov. 7, 2000); U.S. Pat. No. 6,090,556 (Kato; Jul. 18, 2000); U.S. Pat. No. 6,063,568 (Gerdes et al; May 16,2000).

### EXAMPLE 2

LPS-induced sepsis was used in an animal model to detect sepsis. Table 1 shows the mitochondrial DNA (mtDNA) to nuclear DNA (nDNA) ratio in lung and liver tissues 6 hours and 24 hours following administration of LPS to mice.

TABLE 1
Relative Amount Of Mitochondrial DNA In Mouse Tissue
6 h And 24 h Following Administration Of LPS.

| | | | mtDNA/nDNA ratio |
|---|---|---|---|
| treatment | tissue | N | mean ± SD |
| Controls | lung | 6 | 0.29 ± 0.05 |
| LPS-6 h | lung | 6 | 0.23 ± 0.04 |
| LPS-24 h | lung | 6 | 0.25 ± 0.06 |
| Controls | liver | 3 | 2.40 ± 0.99 |
| LPS-24 h | liver | 3 | 2.50 ± 0.22 |

The results indicate a strong trend (P=0.06) toward a lower mtDNA/nDNA ratio in the lung tissue of animals with LPS-induced sepsis.

### CONCLUSION

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the specification, the word “comprising” is used as an open-ended term, substantially equivalent to the phrase “including, but not limited to”, and the word “comprises” has a corresponding meaning. Citation of references herein shall not be construed as an admission that such references are prior art to the present invention. All publications, including but not limited to patents and patent applications, cited in this specification, as well as U.S. provisional application No. 60/393,368, filed Jul. 5, 2002, to which this application claims priority, are incorporated herein by reference as if each individual publication were specifically and individually indicated to be incorporated by reference herein and as though fully set forth herein. The invention includes all embodiments and variations substantially as hereinbefore described and with reference to the examples and drawings.

What is claimed is:

1. A method of diagnosis of a sepsis disease state in a subject in need thereof, comprising determining the relative amount of mitochondrial nucleic acid in a sample from said subject, wherein the determined relative amount of mitochondrial nucleic acid is indicative of the presence of the sepsis disease state in said subject.

2. A method of predicting the risk for a sepsis disease state in a subject in need thereof, comprising determining the relative amount of mitochondrial nucleic acid in a sample from said subject, wherein the determined relative amount of mitochondrial nucleic acid is indicative of the risk for the sepsis disease state in said subject.

3. The method of claim 1 or 2, wherein the subject is suffering from sepsis.

4. A method of monitoring the progression of a sepsis disease state in a subject having sepsis, comprising determining the relative amount of mitochondrial nucleic acid in a sample from said subject at a first time point and determining the relative amount of mitochondrial nucleic acid in a sample from said subject at a second, time point, wherein the difference between determined relative amount of mitochondrial nucleic acid between said first time point and said second time point is indicative of the progression of the sepsis disease state in said subject.

5. The method of claim, further comprising determining the relative amount of mitochondrial nucleic acid in a sample from said subject at subsequent time points.

6. The method of any one of claims 1 through 5, further comprising prognosing or predicting the likely clinical course of sepsis in said subject.

7. A method of determining the efficacy of a therapy for a sepsis disease state, comprising:
i) determining the relative amount of mitochondrial nucleic acid in a sample from a control subject; and
determining the relative amount of mitochondrial nucleic acid in a sample from a test subject, wherein said test subject is administered said therapy;
and wherein the difference between said determined relative amount of mitochondrial nucleic acid in said sample from said test subject and said control subject is indicative of the efficacy of said therapy.

8. The method of any one of claims 1 through 7, wherein said sepsis disease state is a sepsis symptom resulting from a condition selected from the group consisting of gram negative bacterial infection, gram positive bacterial infection, fungal infection, viral infection, protozoan infection, physical trauma, pancreatitis, organ transplantation, hemorrhage, adult respiratory distress syndrome, burn injury, surgery, chemotherapy, and exposure to ionizing radiation.

9. The method of any one of claims 1 through 8, wherein said relative amount of mitochondrial nucleic acid indicates the severity of sepsis or the success of a therapeutic treatment for sepsis.

10. The method of claim 1 through 9, wherein said mitochondrial nucleic acid is determined relative to the amount of nuclear nucleic acid in a cell of said subject.

11. The method of claim 10, wherein said mitochondrial nucleic acid or said nuclear nucleic acid is determined by a polymerase chain reaction.

12. The method of claim 11, wherein said polymerase chain reaction is a quantitative polymerase chain reaction, wherein amplification of the mitochondrial nucleic acid is compared to amplification of a reference nucleic acid.

13. The method of claim 12, wherein said polymerase chain reaction is a real-time polymerase chain reaction wherein an amplification product is detected with a hybridization probe.

14. The method of any one of claims 1 through 13, wherein said subject is diagnosed with sepsis or determined to be at risk for sepsis if said relative amount of mitochondrial nucleic acid falls below a predetermined level.

15. The method of claim 14, wherein said predetermined level is expressed as a ratio of mitochondrial DNA (mtDNA) to nuclear DNA (nDNA) with reference to a standard mtDNA/nDNA ratio set at 1, and wherein said predetermined level is a ratio of 0.45 or less.

16. The method of claim any one of claims 1 through 15, further comprising initiating a treatment for sepsis in said subject when said relative amount of mitochondrial nucleic acid falls below a predetermined level.

17. The method of claim 16, wherein said predetermined level of mitochondrial DNA is expressed as a ratio of mitochondrial DNA (mtDNA) to nuclear DNA (nDNA) with reference to a standard mtDNA/nDNA ratio set at 1, and wherein said predetermined level is a ratio of 0.45 or less.

18. The method of any one of claims 1 through 17 wherein said subject is a human.

19. The method of any one of claims 1 through 18, wherein said subject is selected from the group consisting of a neonate, an immunocompromised individual, and an elderly individual.

20. The method of any one of claims 1 through 19, wherein the subject is a non-human animal.

21. The method of claim 20, wherein said non-human animal is an animal model of sepsis.

22. The method of claim 21, wherein said animal model of sepsis is LPS-induced sepsis.

23. The method of any one of claims 1 through 22, wherein said sample is a peripheral blood sample.

24. The method of any one of claims 1 through 23, wherein the mitochondrial nucleic acid is DNA.

25. The method of any one of claims 1 through 23, wherein the mitochondrial nucleic acid is RNA.

26. The method of claim 25, wherein the RNA is mRNA.

27. A diagnostic kit for use in determining the extent of a sepsis disease state in a subject by determining the relative amount of mitochondrial nucleic acid in a sample from said subject, said kit comprising:
i) a mitochondrial nucleic acid primer; and
ii) a nuclear nucleic acid primer.

28. The kit of claim 27, wherein the mitochondrial nucleic acid is DNA.

29. The kit of claim 27 or 28, wherein the mitochondrial nucleic acid is RNA.

30. The kit of claim 29, wherein the RNA is mRNA.
